(19) United States
(12) Patent Application Publication
(10) Pub. No.: US 2006/0178387 A1
(43) Pub. Date: Aug. 10, 2006

(54) Combined treatment with capecitabine and an epidermal growth factor receptor kinase inhibitor

(76) Inventors: Kaori Fujimoto-Ouchi, Kanagawa (JP); Fumiko Sekiguchi, Kanagawa (JP); Yutaka Tanaka, Kanagawa (JP); Mieko Yanagisawa, Kanagawa (JP)

Correspondence Address: OSI PHARMACEUTICALS, INC., 41 PINELAWN ROAD, MELVILLE, NY, 11747, US

(21) Appl. No.: 11/345,894

(22) Filed: Feb. 2, 2006

Related U.S. Application Data
(60) Provisional application No. 60/650,025, filed on Feb. 4, 2005.

(51) Int. Cl. A61K 31/513

(52) U.S. Cl. 514/269

(57) ABSTRACT

The present invention provides a method for treating tumors or tumor metastases in a patient, comprising administering to the patient simultaneously or sequentially a therapeutically effective amount of an EGFR kinase inhibitor and capecitabine combination, with or without additional agents or treatments, such as other anti-cancer drugs or radiation therapy. The invention also encompasses a pharmaceutical composition that is comprised of an EGFR kinase inhibitor and capecitabine combination in combination with a pharmaceutically acceptable carrier. A preferred example of an EGFR kinase inhibitor that can be used in practicing this invention is the compound erlotinib HCl (also known as TARCEVA®).

### CROSS-REFERENCE TO RELATED APPLICATIONS

This application claims the benefit of U.S. Provisional Application No. 60/650,025, filed Feb. 4, 2005, which is herein incorporated by reference in its entirety.

### BACKGROUND OF THE INVENTION

The present invention is directed to compositions and methods for treating cancer patients. In particular, the present invention is directed to combined treatment of patients with capecitabine and an epidermal growth factor receptor (EGFR) kinase inhibitor.

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), and hormone therapy (e.g., tamoxifen, flutamide).

With the exception of lung cancer, the most prevalent cancer types in the US are colorectal cancer (CRC) and breast cancer, with 130,000 and 183,000 respective new cases documented in 2000. Deaths from CRC and breast cancer combined represent almost one-fifth of all cancer-related deaths in the US. Current treatment options for advanced CRC and metastatic breast cancer (MBC) rely on systemic cytotoxic chemotherapy. The goals of chemotherapy in this setting are to obtain maximum control of symptoms, prevent serious complications and prolong survival, while maintaining or improving quality of life. However, current chemotherapy regimens afford only limited survival benefits, with approximate survival of 6 and 12 months reported in advanced CRC and breast cancer, respectively. Thus, there is a pressing need for therapies that achieve improved progression-free and overall survival in these indications.

Treatment of colorectal cancer depends largely on the size, location and stage of the tumor, whether the malignancy has spread to other parts of the body (metastasis), and on the patient's general state of health. Options include surgical removal of tumors for early stage localized disease, chemotherapy and radiotherapy. However, chemotherapy is currently the only treatment for metastatic disease. 5-fluorouracil is currently the most effective single-agent treatment for advanced colorectal cancer, with response rates of about 10%. Additionally, new agents such as the topoisomerase I inhibitor irinotecan (CPT11), the platinum-based cytotoxic agent oxaliplatin (e.g. ELOXATIN™), capecitabine (XELODA®), and the EGFR kinase inhibitor erlotinib ([6,7-bis(2-methoxyethoxy)-4-quinazolin-4-yl]-(3-ethynylphenyl)amine, e.g. erlotinib HCl, TARCEVA®) have shown promise in treatment.

The epidermal growth factor receptor (EGFR) family comprises four closely related receptors (HER1/EGFR, HER2, HER3 and HER4) involved in cellular responses such as differentiation and proliferation. Over-expression of the EGFR kinase, or its ligand TGF-alpha, is frequently associated with many cancers, including breast, lung, colorectal, ovarian, renal cell, bladder, and head and neck cancers, and is believed to contribute to the malignant growth of these tumors. A specific deletion-mutation in the EGFR gene (EGFRvIII) has also been found to increase cellular tumorigenicity. Activation of EGFR stimulated signaling pathways promote multiple processes that are potentially cancer-promoting, e.g. proliferation, angiogenesis, cell motility and invasion, decreased apoptosis and induction of drug resistance. Increased HER1/EGFR expression is frequently linked to advanced disease, metastases and poor prognosis. For example, in NSCLC and gastric cancer, increased HER1/EGFR expression has been shown to correlate with a high metastatic rate, poor tumor differentiation and increased tumor proliferation.

The development for use as anti-tumor agents of compounds that directly inhibit the kinase activity of the EGFR, as well as antibodies that reduce EGFR kinase activity by blocking EGFR activation, are areas of intense research effort (de Bono J. S. and Rowinsky, E. K. (2002) Trends in Mol. Medicine 8:S19-S26; Dancey, J. and Sausville, E. A. (2003) Nature Rev. Drug Discovery 2:92-313). Several studies have demonstrated, disclosed, or suggested that some EGFR kinase inhibitors might improve tumor cell or neoplasia killing when used in combination with certain other anti-cancer or chemotherapeutic agents or treatments (e.g. Raben, D. et al. (2002) Semin. Oncol. 29:37-46; Herbst, R.S. et al. (2001) Expert Opin. Biol. Ther. 1:719-732; Magne, N., et al. (2003) Clin. Can. Res. 9:47354732; Hidalgo, M. et al. (2003) Oncology 17 (No.11, Suppl.12):11-16; Tanaka, Y. et al. (2003) Proc. Am. Assoc. Cancer Res. 44:930, abstract 4678; Dorligschaw, 0. et al (2003) Proc. Am. Soc. Clin. Oncol. 22:372 (abstract 1494); Magne, N. et al. (2002) British Journal of Cancer 86:819-827; Torrance, C. J. et al. (2000) Nature Med. 6:1024-1028; Gupta, R. A. and DuBois, R. N. (2000) Nature Med. 6:974-975; Tortora, et al. (2003) Clin. Cancer Res. 9:1566-1572; Solomon, B. et al (2003) Int. J. Radiat. Oncol. Biol. Phys. 55:713-723; Krishnan, S. et al. (2003) Frontiers in Bioscience 8, el -13; Huang, S et al. (1999) Cancer Res. 59:1935-1940; Contessa, J. N. et al. (1999) Clin. Cancer Res. 5:405-411; Li, M. et al. Clin. (2002) Cancer Res. 8:3570-3578; Ciardiello, F. et al. (2003) Clin. Cancer Res. 9:1546-1556; Ciardiello, F. et al. (2000) Clin. Cancer Res. 6:3739-3747; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther.3:367-380; Bulgaru, A. M. et al. (2003) Expert Rev. Anticancer Ther.3:269-279; Dancey, J. and Sausville, E. A. (2003) Nature Rev. Drug Discovery 2:92-313; Kim, E. S. et al. (2001) Current Opinion Oncol. 13:506-513; Arteaga, C. L. and Johnson, D. H. (2001) Current Opinion Oncol. 13:491-498; Ciardiello, F. et al. (2000) Clin. Cancer Res. 6:2053-2063; Patent Publication Nos: US 2003/0108545; US 2002/0076408; and US 2003/0157104; and International Patent Publication Nos: WO 99/60023; WO 01/12227; WO 02/055106; WO 03/088971; WO 01/34574; WO 01/76586; WO 02/05791; and WO 02/089842).

Erlotinib (e.g. erlotinib HCl, also known as TARCEVA® or OSI-774) is an orally available inhibitor of EGFR kinase. In vitro, erlotinib has demonstrated substantial inhibitory activity against EGFR kinase in a number of human tumor cell lines, including colorectal and breast cancer (Moyer J. D. et al. (1997) Cancer Res. 57:4838), and preclinical evaluation has demonstrated activity against a number of EGFR-expressing human tumor xenografts (Pollack, V. A. et al (1999) J. Pharmacol. Exp. Ther. 291:739). More recently, erlotinib has demonstrated promising activity in phase I and II trials in a number of indications, including head and neck cancer (Soulieres, D., et al. (2004) J. Clin. Oncol. 22:77), NSCLC (Perez-Soler R, et al. (2001) Proc. Am. Soc. Clin. Oncol. 20:310a, abstract 1235), CRC (Oza, M., et al. (2003) Proc. Am. Soc. Clin. Oncol. 22:196a, abstract 785) and MBC (Winer, E., et al. (2002) Breast Cancer Res. Treat. 76:5115a, abstract 445). In a phase III trial, erlotinib monotherapy significantly prolonged survival, delayed disease progression and delayed worsening of lung cancer-related symptoms in patients with advanced, treatment-refractory NSCLC (Shepherd, F. et al. (2004) J. Clin. Oncology, 22:14S (July 15 Supplement), Abstract 7022).

Oral capecitabine is a highly effective fluoropyrimidine, which generates 5-fluorouracil (5-FU) preferentially in tumor tissue by exploitation of the increased activity of thymidine phosphorylase (TP) in tumors compared with normal tissue. 5-FU is deactivated by the enzyme dihydropyrimidine dehydrogenase (DPD), and the TP:DPD ratio has been shown to correlate with susceptibility to capecitabine in human tumor xenograft models. Capecitabine has demonstrated consistent and impressive activity in patients with chemo-naïve and pretreated advanced breast cancer (Reichardt, P., et al. (2003) Ann. Oncol. 14:1227), and advanced CRC (Twelves, C. (2002) Eur. J. Cancer 38 (Suppl 2):15).

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current chemotherapies possess such an ideal profile. Instead, most possess very narrow therapeutic indexes. Furthermore, cancerous cells exposed to slightly sub-lethal concentrations of a chemotherapeutic agent will very often develop resistance to such an agent, and quite often cross-resistance to several other antineoplastic agents as well.

Thus, there is a need for more efficacious treatment for neoplasia and other proliferative disorders. Strategies for enhancing the therapeutic efficacy of existing drugs have involved changes in the schedule for their administration, and also their use in combination with other anticancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone).

Target-specific therapeutic approaches, such as erlotinib, are generally associated with reduced toxicity compared with conventional cytotoxic agents, and therefore lend themselves to use in combination regimens. Promising results have been observed in phase I/II studies of erlotinib in combination with bevacizumab (Mininberg, E. D., et al. (2003) Proc. Am. Soc. Clin. Oncol. 22:627a, abstract 2521) and gemcitabine (Dragovich, T., (2003) Proc. Am. Soc. Clin. Oncol. 22:223a, abstract 895). Recent data in NSCLC phase III trials have shown that first-line erlotinib or gefitinib in combination with standard chemotherapy did not improve survival (Gatzemeier, U., (2004) Proc. Am. Soc. Clin. Oncol. 23:617 (Abstract 7010); Herbst, R. S., (2004) Proc. Am. Soc. Clin. Oncol. 23:617 (Abstract 7011); Giaccone, G., et al. (2004) J. Clin. Oncol. 22:777; Herbst, R., et al. (2004) J. Clin. Oncol. 22:785). However, pancreatic cancer phase III trials have shown that first-line erlotinib in combination with gemcitabine did improve survival (OSI Pharmaceuticals/Genentech/Roche Pharmaceuticals Press Release, 9/20/04). Capecitabine has been used successfully in combination therapy regimens (O'Shaughnessy, J., et al. (2002) J. Clin. Oncol. 20:2812; Scheithauer, W., et al (2003) J. Clin. Oncol. 21:1307), and preclinical studies have demonstrated or suggested potential supra-additive activity with combination regimens comprising capecitabine and any one of a number of anticancer therapies, including gefitinib (Magne, N., et al. (2003) Clin. Cancer Res. 9:4735), docetaxel and paclitaxel (Fujimoto-Ouchi, K., et al. (2001) Clin. Cancer Res. 7:1079; Sawada, N., et al. (1998) Clin. Cancer Res. 4:1013), cyclophosphamide (Endo, M., et al. (1999) Int. J. Cancer 83:127) and radiotherapy (Sawada, N., et al (1999) Clin. Cancer Res. 5:2948). In addition, at least additive antitumor activity has been demonstrated with capecitabine and trastuzumab, an agent specifically targeting the HER2 receptor (Fujimoto-Ouchi, K., et al. (2002) Cancer Chemother. Pharmacol. 49:211).

However, there remains a critical need for improved treatments for colorectal, breast, and other cancers. This invention provides anti-cancer combination therapies that reduce the dosages for individual components required for efficacy, thereby decreasing side effects associated with each agent, while maintaining or increasing therapeutic value. The invention described herein provides new drug combinations, and methods for using drug combinations in the treatment of colorectal and other cancers.

### SUMMARY OF THE INVENTION

The present invention provides a method for treating tumors or tumor metastases in a patient, comprising administering to the patient simultaneously or sequentially a therapeutically effective amount of an EGFR kinase inhibitor and capecitabine combination, with or without additional agents or treatments, such as other anti-cancer drugs or radiation therapy.

The invention also encompasses a pharmaceutical composition that is comprised of an EGFR kinase inhibitor and capecitabine combination in combination with a pharmaceutically acceptable carrier.

A preferred example of an EGFR kinase inhibitor that can be used in practicing this invention is the compound erlotinib HCl (also known as TARCEVA®).

### BRIEF DESCRIPTION OF THE FIGURES

FIG. 1: Dose-dependent antitumor activity of erlotinib in the human colon cancer xenograft model (LoVo). Mean values±SD of tumor volume (cm3). Vehicle (diamonds), erlotinib at 50 (crosses) 75 (triangles), 100 (squares) or 125 (circles) mg/kg. *P<0.05 vs vehicle control.

FIG. 2: Antitumor activity of erlotinib (100 mg/kg/day) as a single agent in five human cancer xenograft models (HT-29, LoVo, KPL-4, MAXF401 and A-43 1). Mean values±SD of tumor volume (cm³). Vehicle (diamonds), and erlotinib (squares). *P<0.05 vs vehicle control.

FIG. 3: Enhanced antitumor effect of erlotinib (100 mg/kg/day) in combination with capecitabine (359 mg/kg/day) on tumor volume in LoVo human tumor xenograft models. Mean values±SD of tumor volume (cm³). Vehicle (diamonds), capecitabine alone (circles), erlotinib alone (squares), capecitabine in combination with erlotinib (triangles). *P<0.05.

FIG. 4: Enhanced antitumor effect of erlotinib (100 mg/kg/day) in combination with capecitabine (90 mg/kg/day) on tumor volume in KPL-4 human tumor xenograft models. Mean values±SD of tumor volume (cm³). Vehicle (diamonds), capecitabine alone (circles), erlotinib alone (squares), capecitabine in combination with erlotinib (triangles). *P<0.05.

FIG. 5: Enhanced antitumor effect of erlotinib (100 mg/kg/day) in combination with capecitabine (359 mg/kg/day) on tumor volume in A-431 human tumor xenograft models. Mean values±SD of tumor volume (cm³). Vehicle (diamonds), capecitabine alone (circles), erlotinib alone (squares), capecitabine in combination with erlotinib (triangles). **P<0.01.

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The data presented in the Examples herein below demonstrate that co-administration of capecitabine with an EGFR kinase inhibitor is effective for treatment of patients with advanced cancers, such as colorectal cancer or breast cancer. Accordingly, the present invention provides a method for treating tumors or tumor metastases in a patient, including colorectal cancer, breast cancer, or epidermal cancer tumors, comprising administering to said patient simultaneously or sequentially a therapeutically effective amount of an EGFR kinase inhibitor and capecitabine combination.

The present invention further provides a method for treating tumors or tumor metastases in a patient, comprising administering to the patient simultaneously or sequentially a therapeutically effective amount of an EGFR kinase inhibitor and capecitabine combination, and in addition, one or more other cytotoxic, chemotherapeutic or anti-cancer agents, or compounds that enhance the effects of such agents.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6MP), 6-thiocguanine (6TG), cytarabine (Ara-C), 5-fluorouracil (5-FU), capecitabine (e.g.XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®)) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®)), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides a method for treating tumors or tumor metastases in a patient, comprising administering to said patient simultaneously or sequentially a therapeutically effective amount of an EGFR kinase inhibitor and capecitabine combination, and in addition, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6- (1-methylethyl)-L-lysyl -L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4, 20-nitro-3-(trifluoromethyl) phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides a method for treating tumors or tumor metastases in a patient, comprising administering to said patient simultaneously or sequentially a therapeutically effective amount of an EGFR kinase inhibitor and capecitabine combination, and in addition one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥ integrins, and subtypes thereof, e.g. cilengitide (EME 121974), or the anti-integrin antibodies, β₆ integrins, and subtypes thereof, e.g., cilengitide (EMD 121974(, or the anti-integrin antibodies, such as for example αᵥβ₃ specific humanized antibodies (e.g. VITAXIN®); factors such as IFN-alpha (U.S. Pat. Nos. 41530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-470 1; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP4, MMP-5, MMP-6, MMP-7, MMP-8, MMP-10, MMP-I 1, MMP-12, and MMP-13).

The present invention further provides a method for treating tumors or tumor metastases in a patient, comprising administering to the patient simultaneously or sequentially a therapeutically effective amount of an EGFR kinase inhibitor and capecitabine combination, and in addition one or more tumor cell pro-apoptotic or apoptosis-stimulating agents.

The present invention further provides a method for treating tumors or tumor metastases in a patient, comprising administering to said patient simultaneously or sequentially a therapeutically effective amount of an EGFR kinase inhibitor and capecitabine combination, and in addition one or more signal transduction inhibitors.

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); ras inhibitors; raf inhibitors (e.g. BAY 43-9006, Onyx Pharmaceuticals/Bayer Pharmaceuticals); MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Patent Nos. 5,587,458, 5,877,305, 6,465,449 and 6,541,481.

The present invention further thus provides a method for treating tumors or tumor metastases in a patient, comprising administering to said patient simultaneously or sequentially a therapeutically effective amount of an EGFR kinase inhibitor and capecitabine combination, and in addition an anti-HER2 antibody or an immunotherapeutically active fragment thereof.

The present invention further provides a method for treating tumors or tumor metastases in a patient, comprising administering to said patient simultaneously or sequentially a therapeutically effective amount of an EGFR kinase inhibitor and capecitabine combination, and in addition one or more additional anti-proliferative agents.

Additional antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The present invention further provides a method for treating tumors or tumor metastases in a patient, comprising administering to the patient simultaneously or sequentially a therapeutically effective amount of an EGFR kinase inhibitor and capecitabine combination, and in addition a COX II (cyclooxygenase II ) inhibitor. Examples of useful COX-II inhibitors include alecoxib (e.g. CELEBREX™), valdecoxib, and rofecoxib.

The present invention further provides a method for treating tumors or tumor metastases in a patient, comprising administering to the patient simultaneously or sequentially a therapeutically effective amount of an EGFR kinase inhibitor and capecitabine combination, and in addition treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides a method for treating tumors or tumor metastases in a patient, comprising administering to the patient simultaneously or sequentially a therapeutically effective amount of an EGFR kinase inhibitor and capecitabine combination, and in addition treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

The present invention further provides a method for reducing the side effects caused by the treatment of tumors or tumor metastases in a patient with an EGFR kinase inhibitor or capecitabine, comprising administering to the patient simultaneously or sequentially a therapeutically effective amount of an EGFR kinase inhibitor and capecitabine combination, in amounts that are effective to produce an additive, or a superadditive or synergistic antitumor effect, and that are effective at inhibiting the growth of the tumor.

The present invention further provides a method for the treatment of cancer, comprising administering to a subject in need of such treatment (i) an effective first amount of an EGFR kinase inhibitor, or a pharmaceutically acceptable salt thereof; and (ii) an effective second amount of capecitabine. In this method the cancer can be any of those referred to herein below, including colorectal, breast or epidermal cancers.

The present invention also provides a method for the treatment of cancer, comprising administering to a subject in need of such treatment (i) a sub-therapeutic first amount of an EGFR kinase inhibitor, or a pharmaceutically acceptable salt thereof; and (ii) a sub-therapeutic second amount of capecitabine. In this method the cancer can be any of those referred to herein below, including colorectal, breast or epidermal cancers.

The present invention also provides a method for the treatment of cancer, comprising administering to a subject in need of such treatment (i) an effective first amount of an EGFR kinase inhibitor, or a pharmaceutically acceptable salt thereof; and (ii) a sub-therapeutic second amount of capecitabine. In this method the cancer can be any of those referred to herein below, including colorectal, breast or epidermal cancers.

The present invention also provides a method for the treatment of cancer, comprising administering to a subject in need of such treatment (i) a sub-therapeutic first amount of an EGFR kinase inhibitor, or a pharmaceutically acceptable salt thereof; and (ii) an effective second amount of capecitabine. In this method the cancer can be any of those referred to herein below, including colorectal, breast or epidermal cancers.

In the preceding methods the order of administration of the first and second amounts can be simultaneous or sequential, i.e. capecitabine can be administered before the EGFR kinase inhibitor, after the EGFR inhibitor, or at the same time as the EGFR kinase inhibitor.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

Additionally, the present invention provides a pharmaceutical composition comprising an EGFR inhibitor and capecitabine in a pharmaceutically acceptable carrier.

As used herein, the term “patient” preferably refers to a human in need of treatment with an EGFR kinase inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an EGFR kinase inhibitor.

In a preferred embodiment, the patient is a human in need of treatment for cancer, or a precancerous condition or lesion. The cancer is preferably any cancer treatable, either partially or completely, by administration of an EGFR kinase inhibitor. The cancer may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

For purposes of the present invention, “co-administration of” and “co-administering” capecitabine with an EGFR kinase inhibitor (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. capecitabine can be administered prior to, at the same time as, or subsequent to administration of the EGFR kinase inhibitor, or in some combination thereof. Where the EGFR kinase inhibitor is administered to the patient at repeated intervals, e.g., during a standard course of treatment, capecitabine can be administered prior to, at the same time as, or subsequent to, each administration of the EGFR kinase inhibitor, or some combination thereof, or at different intervals in relation to the EGFR kinase inhibitor treatment, or in a single dose prior to, at any time during, or subsequent to the course of treatment with the EGFR kinase inhibitor.

The EGFR kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the EGFR kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of EGFR kinase inhibitor being used (for example, small molecule, antibody, RNAi, ribozyme or antisense construct), and the medical judgement of the prescribing physician as based, e.g., on the results of published clinical studies.

The amount of EGFR kinase inhibitor administered and the timing of EGFR kinase inhibitor administration will depend on the type (species, gender, age, weight, etc.) and condition of the patient being treated, the severity of the disease or condition being treated, and on the route of administration. For example, small molecule EGFR kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based EGFR kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The EGFR kinase inhibitors and capecitabine can be administered either separately or together by the same or different routes, and in a wide variety of different dosage forms. For example, the EGFR kinase inhibitor is preferably administered orally or parenterally, whereas capecitabine is preferably administered orally or parenterally. Where the EGFR kinase inhibitor is erlotinib HCl (TARCEVA), oral administration is preferable. Where the capecitabine is XELODA®, oral administration is preferable. Both the EGFR kinase inhibitor and capecitabine can be administered in single or multiple doses.

The EGFR kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The EGFR kinase inhibitor and capecitabine can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous EGFR kinase inhibitors should be selected so as to avoid denaturation and/or degradation and loss of biological activity of the inhibitor.

Methods of preparing pharmaceutical compositions comprising an EGFR kinase inhibitor are known in the art, and are described, e.g. in International Patent Publication No. WO 01/34574. Methods of preparing pharmaceutical compositions comprising capecitabine are also well known in the art (e.g. see XELODA®, Roche Pharmaceuticals). In view of the teaching of the present invention, methods of preparing pharmaceutical compositions comprising both an EGFR kinase inhibitor and capecitabine will be apparent from the above-cited publications and from other known references, such as Remington's Pharmaceutical Sciences, Mack Publishing Company, Easton, Pa., 18^{th} edition (1990).

For oral administration of EGFR kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous EGFR kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising either an EGFR kinase inhibitor or capecitabine in about 0.1% (w/v) to about 5% (w/v) concentration can be prepared.

For veterinary purposes, the active agents can be administered separately or together to animals using any of the forms and by any of the routes described above. In a preferred embodiment, the EGFR kinase inhibitor is administered in the form of a capsule, bolus, tablet, liquid drench, by injection or as an implant. As an alternative, the EGFR kinase inhibitor can be administered with the animal feedstuff, and for this purpose a concentrated feed additive or premix may be prepared for a normal animal feed. The capecitabine is preferably administered in the form of liquid drench, by injection or as an implant. Such formulations are prepared in a conventional manner in accordance with standard veterinary practice.

The present invention further provides a kit comprising a single container comprising both an EGFR kinase inhibitor and capecitabine. The present invention further provides a kit comprising a first container comprising an EGFR kinase inhibitor and a second container comprising capecitabine. In a preferred embodiment, the kit containers may further include a pharmaceutically acceptable carrier. The kit may further include a sterile diluent, which is preferably stored in a separate additional container. The kit may further include a package insert comprising printed instructions directing the use of the combined treatment as a method for treating cancer.

As used herein, the term “EGFR kinase inhibitor” refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGF receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors that include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl) amine (also known as OSI-774, erlotinib, or TARCEVA® (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:48384848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate ; GSK); and gefitinib (also known as ZD1839 or IRESSATM; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, TARCEVA®), or other salt forms (e.g. erlotinib mesylate).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1: 1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX447 (Medarex/Merck KgaA).

Additional antibody-based EGFR kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against EGFR can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Nati. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-EGFR single chain antibodies. Antibody-based EGFR kinase inhibitors useful in practicing the present invention also include anti-EGFR antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to EGFR.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-EGFR antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494498; Hannon, G. J. (2002) Nature 418:244-251; McManus, M. T. and Sharp, P. A. (2002) Nature Reviews Genetics 3:737-747; Bremmelkamp, T.R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GWU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

The invention also encompasses a pharmaceutical composition that is comprised of an EGFR kinase inhibitor and capecitabine combination in combination with a pharmaceutically acceptable carrier.

Preferably the composition is comprised of a pharmaceutically acceptable carrier and a non-toxic therapeutically effective amount of an EGFR kinase inhibitor compound and capecitabine combination (including pharmaceutically acceptable salts of each component thereof).

Moreover, within this preferred embodiment, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of an EGFR kinase inhibitor compound and capecitabine combination (including pharmaceutically acceptable salts of each component thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present invention comprise an EGFR kinase inhibitor compound and capecitabine combination (including pharmaceutically acceptable salts of each component thereof) as active ingredient, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by an EGFR kinase inhibitor compound and capecitabine combination (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an EGFR kinase inhibitor compound and capecitabine combination (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and an EGFR kinase inhibitor compound and capecitabine combination (including pharmaceutically acceptable salts of each component thereof). An EGFR kinase inhibitor compound and capecitabine combination (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise an EGFR kinase inhibitor compound and capecitabine in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an EGFR kinase inhibitor compound and capecitabine combination (including pharmaceutically acceptable salts of each component thereof) of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an EGFR kinase inhibitor compound and capecitabine combination (including pharmaceutically acceptable salts of each component thereof) may also be prepared in powder or liquid concentrate form.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Introduction

The current study examined the antitumor activity and tolerability of erlotinib in combination with capecitabine in human colorectal, breast and epidermal cancer xenograft models. Further aims of the study were to examine the effects of single-agent erlotinib therapy on tumor growth, and on TP (thymidine phosphorylase) and DPD (dihydropyrimidine dehydrogenase) levels in tumor tissue.

Materials and Methods

Test Agents

Erlotinib (F. Hoffman-La Roche, Nutley, N.J.), as the hydrochloride salt, was provided as a fine powder. After suspension in vehicle (0.2% (w/v) carboxymethylcellulose containing 0.1% (v/v) Tween 80), erlotinib was sonicated for 5 minutes and homogenized for 7 minutes prior to administration. Capecitabine (F. Hoffman-La Roche, Nutley, N.J.) was provided as a powder and suspended in vehicle (40 mM citrate buffer containing 5% (w/v) gum arabic, pH 6.0).

Animals

Male and female, 4-6-weeks old BALB/c nu/nu mice were obtained from Nippon Clea (Tokyo, Japan). All animals were allowed to acclimatize and recover from shipping-related stress for 1 week prior to the study. The health of the mice was monitored daily by observation.

Chlorinated water and irradiated food were provided ad libitum, and the animals were kept in a 12-hour light and dark cycle. All animal experiments were in accordance with the Guidelines for the Care and Use of Laboratory Animals in the Nippon Roche Research Center.

Cell Lines and Culture Conditions

LoVo (human colon cancer) cells (American Type Culture Collection [ATCC], Rockville, Md.) were maintained in Ham's F-12 medium supplemented with 20% (v/v) fetal bovine serum (FBS). HT-29 (human colon cancer) cells (ATCC) were maintained in McCoy's Sa medium supplemented with 10% (v/v) FBS. A-431 (human vulval epidermal cancer) cells (ATCC) were maintained in Dulbecco's modified Eagle's medium (DMEM) nutrient mixture containing 4 mM L-glutamine, 18 mM sodium bicarbonate, 23 mM glucose and 10% (v/v) FBS. KPL-4 (human inflammatory breast cancer) cells (Kurebayashi, J., et al. (1999) Br. J. Cancer 79: 707) were kindly provided by Dr J. Kurebayashi (Kawasaki Medical School, Kurashiki, Japan), and were maintained in DMEM containing 10% (v/v) FBS. MAXF401 (human breast cancer) cells were kindly provided by Dr Prof. H. H. Fiebig (University of Freiburg, Freiburg, Germany), and were maintained in BALB/c nu/nu mice by inoculation subcutaneously (s.c.).

Tumor-Growth Inhibition Studies in vivo

Suspensions of LoVo (5×10⁶ cells/mouse), HT-29 (5 x 106 cells/mouse) and A431 (8×10⁶ cells/mouse) cells were inoculated s.c. into the right flank of the mice. A piece of MAXF401 was transplanted s.c. into the right flank of female mice. A suspension of KPL-4 cells (1×10⁷ cells/mouse) was orthotopically transplanted into the second mammary fat pad of female mice. Several weeks after tumor inoculation, mice were selected and randomized into control and treatment groups. Experiments were started when tumor volumes reached approximately 0.1-0.3 cm³. To evaluate the antitumor activity and tolerability of erlotinib and capecitabine, tumor volume and body weight were assessed twice a week. The tumor volumes were estimated using the equation V=ab²/2, where a and b are tumor length and width, respectively. Gastrointestinal (GI) toxicity was estimated by observing the feces and by detecting fecal occult blood using a test kit (Shionogi Pharma Co., Osaka, Japan). Peripheral blood leukocyte counts were performed to evaluate bone marrow toxicity.

Treatment of Animals

In the dose-response response study, randomized groups of mice (n=5) bearing LoVo tumors were treated with vehicle control or erlotinib at doses of 50, 75, 100 or 125 mg/kg once daily for 2 weeks.

Cohorts of mice bearing LoVo, HT-29, KPL-4, MAXF401 or A-431 tumors were each randomized to groups of five or six mice and treated with erlotinib 100 mg/kg or vehicle control once daily for two weeks.

In the combination studies, mice bearing LoVo or A-431 tumors were each randomized into groups of six mice and treated with erlotinib 100 mg/kg (80% MTD), capecitabine 359 mg/kg (66% MTD) or their respective vehicle controls, or a combination of erlotinib 100 mg/kg and capecitabine 359 mg/kg or combined vehicle control, once daily for 2 weeks (Table 1). Mice bearing KPL-4 tumors were randomized into groups of five and treated with erlotinib 100 mg/kg (80% MTD), capecitabine 90 mg/kg (66% MTD in this model) or their respective vehicle controls, or a combination of erlotinib 100 mg/kg and capecitabine 90 mg/kg or combined vehicle control, once daily for 2 weeks.

In all animals, erlotinib, capecitabine and vehicle controls were administered orally, using a 1 ml syringe and a gavage needle for mice.

TP and DPD Protein Levels in Tumor Tissues

Tumor samples were collected on the day following the final treatment and immediately frozen in liquid nitrogen and stored at −80° C. prior to assay. Tumor tissues were homogenized in phosphate buffered saline (PBS) and centrifuged at 10,000 x g for 20 minutes at 4° C. The protein concentration of the supernatant was determined using a DC Protein Assay Kit (Bio-Rad Laboratories, Hercules, Calif.). The levels of TP and DPD were measured by sandwich enzyme-linked immunosorbent assay (ELISA) with monoclonal antibodies specific to human TP and DPD, as described previously (Nishida, M., (1996) Biol. Pharm. Bull. 19:1407; Mori, K., et al. (2000) Int. J. Oncol. 17: 33).

In the LoVo model, TP and DPD up-regulation was confirmed immunohistochemically, using a polyclonal anti-human TP antibody number 6 (prepared at the Nippon Roche Research Center) and anti-DPD monoclonal antibody (2H9- lb, Roche Diagnostics Ltd, Nutley, N.J.) (Komuro, Y., et al. (2003) Hepato-Gastroenterology 50:906). Polyclonal anti-human TP antibody number 6, obtained by rabbit immunization, was used for immunohistochemical analysis. Antigen was purified from recombinant TP protein. Formalin-fixed, paraffin-embedded specimen sections were dewaxed in xylene and dehydrated by passage through a graded ethanol series to tap water. Antigen retrieval was achieved with steaming in 10 mM citrate buffer (pH 6.0) for 20 minutes followed by 20 minutes cooling down at room temperature. After blocking with 0.3% hydrogen peroxide in methanol, the sections were further blocked for 30 minutes with 3% skimmed milk. The sections were incubated overnight (4° C.) with anti-TP polyclonal antibody number 6. Sections were subsequently incubated with biotinylated rat anti-rabbit immunoglobulins followed by avidin-peroxidase reagent. Reactivity was visualized using 3,3′-diaminobenzidine as the substrate.

Statistical Analysis

The Mann-Whitney U test was used to determine differences in tumor volume, body weight and tumor TP and DPD concentrations between the groups.

Results

Effects of Erlotinib on Established Human Tumor Xenografts

Dose Response Study in LoVo

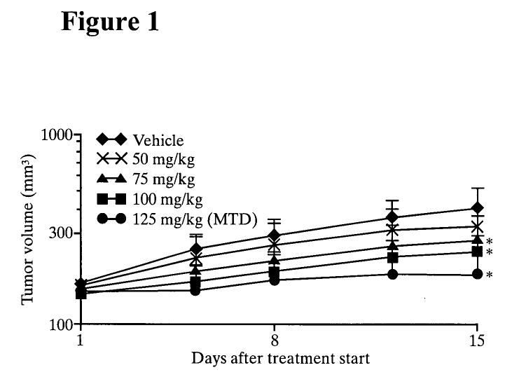

Erlotinib demonstrated dose-dependent antitumor activity against LoVo tumors at doses ranging from 50 to 125 mg/kg/day (FIG. 1). On day 15, after 14 days' treatment with erlotinib, tumor inhibition rates of 52%, 62% and 85% were observed with erlotinib doses of 75, 100 and 125 mg/kg, respectively. The 50 mg/kg dose of erlotinib did not show significant antitumor activity. Substantial weight loss (>20% of the body weight at start of treatment) was not observed at any of the doses tested. No toxic deaths were observed.

In a separate experiment, erlotinib 250 mg/kg/day was shown to be lethal in mice bearing LoVo tumors and in mice bearing A431 tumors (data not shown). Therefore 125 mg/kg/day erlotinib formulated in carboxymethylcellulose/Tween 80 was identified as the maximum tolerated dose (MTD).

Antitumor Activity as a Single Agent In Five Xenograft Models

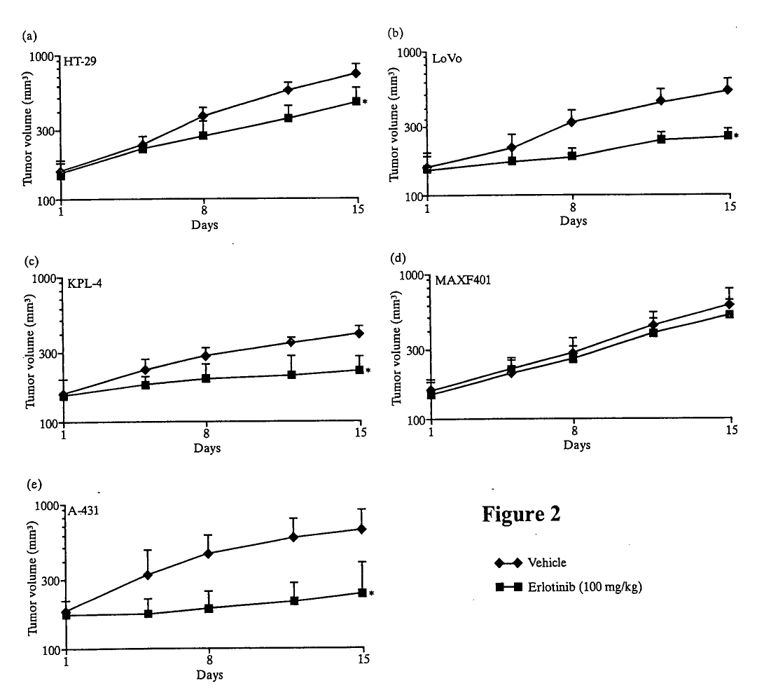

Erlotinib at a dose of 100 mg/kg/day (80% of MTD) was administered to mice bearing established HT-29, LoVo, KPL4, MAXF401 or A431 tumors. Significant tumor-growth inhibition was observed in the HT-29, LoVo, KPL4, and A431 models (FIG. 2). Some inhibition of MAXF401 tumor growth was observed after 15 days, but was not statistically significant. At day 15, mean tumor-growth inhibition in HT-29, LoVo, KPL4, MAXF401, and A431 models was 46%, 74%, 71%, 20%, and 85%, respectively.

Combination Activity of Erlotinib and Capecitabine in Colon, Breast and Epidermal Tumor Xenografts

Combination therapy with erlotinib and capecitabine was examined in the three erlotinib-sensitive tumor models: LoVo, KPL4 and A43 1.

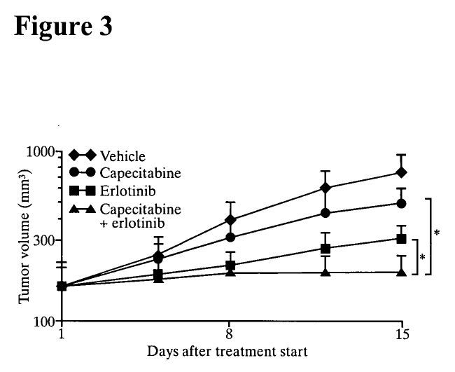

In the LoVo colorectal cancer model, 100 mg/kg/day of erlotinib (80% of MTD) and 359 mg/kg of capecitabine (67% of MTD (Ishikawa, T., et al. (1998) Cancer Res. 58: 685)) were administered separately and in combination, once daily for 14 days starting on day 17 following tumor inoculation. Combined treatment with erlotinib and capecitabine achieved significantly increased tumor inhibition compared with either capecitabine or erlotinib administered as single agents (FIG. 3; P<0.05). On day 15 after treatment started, inhibition of tumor growth was 43%, 74% and 95% in the capecitabine, erlotinib and combination treatment groups, respectively (Table 2). Furthermore, the antitumor activity of the combination treatment was greater than that of capecitabine at the MTD (539 mg/kg, 56%).

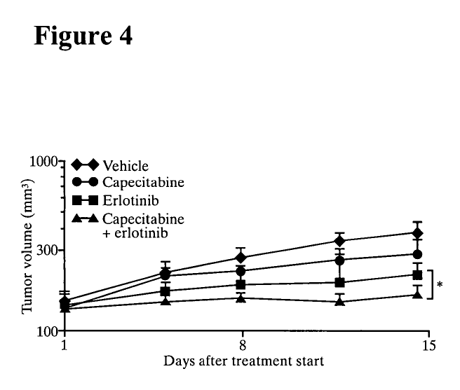

In the KPL4 breast cancer model, 100 mg/kg/day of erlotinib (80% of MTD) and 90 mg/kg of capecitabine (67% of MTD for this model only (Fujimoto-Ouchi, K., et al. (2002) Cancer Chemother. Pharmacol. 49:211)) were administered once daily for 14 days, commencing on day 16 following tumor inoculation. Combined treatment with erlotinib and capecitabine achieved significantly increased tumor inhibition compared with capecitabine administered as a single agent (FIG. 4; P<0.05). Tumor-growth inhibition on day 15 was 36%, 71% and 88%, in the capecitabine, erlotinib and the combination groups, respectively (Table 2). A combination regimen comprising 75 mg/kg of erlotinib (60% of MTD) and 90 mg of capecitabine (66% of MTD) achieved 82% inhibition of tumor growth, which was significantly increased compared with single-agent erlotinib (45%; data not shown) or capecitabine.

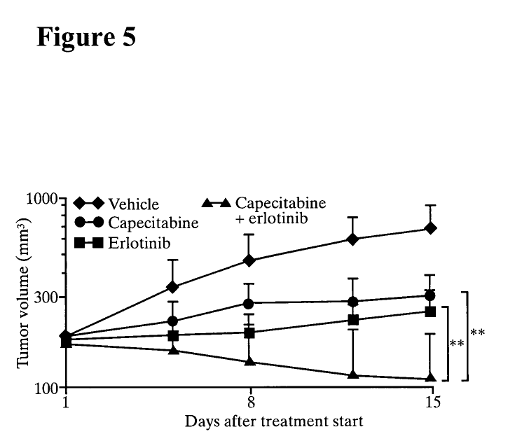

In the A431 epidermal cancer model, 100 mg/kg/day of erlotinib (80% of MTD) and 359 mg/kg of capecitabine (66% of MTD) were administered once daily for 14 days, commencing 12 days after tumor inoculation (FIG. 5). At day 15, a highly significant increase in tumor-growth inhibition was observed with the combination compared with single-agent therapies, with tumor-growth inhibition of 76%, 85% and 112% documented in the capecitabine, erlotinib and combination groups, respectively (Table 2). As well as for the LoVo model, the antitumor activity of the combination was more potent than that of capecitabine at the MTD dose (539mg/kg, data not shown) in the A431 model.

In all of the models tested, combination treatment with erlotinib and capecitabine was not associated with significant toxicity, and there was no mean body weight loss greater than 20% of pretreatment body weight. Intestinal toxicity (diarrhea) and myelotoxicity (peripheral blood leukocyte count) were further evaluated in a separate experiment in mice bearing LoVo tumors. These mice were treated with erlotinib 100 mg/kg/day and 359 mg/kg of capecitabine once daily for 14 days. On day 15, no intestinal toxicity (assessed by fecal-form observation and occult blood test) and no significant differences between the peripheral blood leukocyte counts were observed in any group compared with the control values (Table 3).

Effects of Erlotinib on Tumor Enzyme Levels

The effect of erlotinib treatment on TP and DPD levels in tumor tissue was examined. Tumor TP and DPD levels are summarized in Table 4. In the LoVo model, up-regulation of TP and DPD was observed at 100 mg erlotinib/kg/day (Table 4). In this model, TP up-regulation was also confirmed immunohistochemically. Immunohistochemical staining of TP was very strong in LoVo tumors removed from mice treated with erlotinib, whereas tumor samples from vehicle-treated animals did not express TP (data not shown).

In the MAXF401 and HT-29 models, significant (P<0.05) differences in TP levels were observed in tumors from animals treated with erlotinib 100 mg/kg compared with vehicle control, but there were no significant differences in DPD levels in these models. No significant differences were observed in TP or DPD levels in the KPL4 model. In the A431 model, TP expression was significantly up-regulated by the administration of erlotinib 100 mg/kg, but DPD was unaffected. In the A431 model, the TP:DPD ratio was therefore increased (1.7-fold increase, P<0.01).

Discussion

Human tumor xenograft models are commonly used for the evaluation of anticancer agents. In the current study, five human tumor xenograft models were used to evaluate the activity and tolerability of erlotinib, administered both alone and in combination with capecitabine.

Erlotinib demonstrated a dose-dependent antitumor effect when administered to mice bearing LoVo colon tumors at doses ranging from 50-125 mg/kg/day. At the lowest dose of erlotinib showing a significant response (75 mg/kg/day), tumor inhibition of approximately 50% was observed. Erlotinib was well tolerated at these doses, with no deaths recorded and mean body weight maintained (≧80%) in all groups. Additional data showed that erlotinib 250 mg/kg/day was lethal in this model (data not shown), indicating that the MTD for erlotinib is 125 mg/kg/day, when formulated as a suspension in carboxymethylcellulose/Tween 80.

In mice bearing human colon (LoVo), breast (KPL4), epidermal (A431) tumors, erlotinib monotherapy resulted in highly significant inhibition of tumor growth compared with vehicle control. The significant antitumor activity of erlotinib in these tumor xenograft models supports the use of erlotinib for the treatment of tumors expressing HER1/EGFR, such as breast (Klijn, J. G., et al. (1992) Endocr. Rev. 13:3), colon (Messa, C., et al. (1998) Acta Oncol. 37:285), head and neck (Grandis, J. R., et al.(1996) Cancer 78:1284) and NSCLC (Rusch, V., et al. (1997) Clin. Cancer Res. 3:515) cancers.

It has been postulated that HER1/EGFR inhibition may potentiate the activity of anticancer agents by inhibiting the ability of cells to repair chemo- or radiotherapy-induced damage (Woodburn, J. R. (1999) Pharmacol. Ther. 82:241; Kastan, M. B. (1997) Am. Soc. Clin. Oncol. Educational Book. W. B. Saunders, p15). Furthermore, HER1/EGFR inhibition may limit the development of resistance to conventional anticancer therapies and facilitate their use at reduced doses, thereby reducing the incidence and/or intensity of the adverse events associated with these therapies. In the current study, erlotinib and capecitabine were investigated at doses corresponding to 80% and 67% of their respective MTDs, and the combination was well tolerated, with no significant increase in toxicity compared with the constitutive single agents. In the A431 and LoVo models, the combination of agents showed more potent antitumor activity than capecitabine alone at the MTD. Such results are suggestive that the combination of erlotinib with capecitabine may show clinical benefit in patients. Furthermore, the oral administration schedules for both erlotinib and capecitabine offer the potential for significantly improved patient convenience and quality of life compared with conventional intravenous chemotherapy-based regimens.

In accordance with the established clinical efficacy of capecitabine in the management of colorectal and breast cancer [Twelves, C. (2002) Eur. J. Cancer 38 (Suppl 2):15; Blum, J. L., et al. (2001) Cancer 92:1759), capecitabine administered as a single agent significantly inhibited tumor-growth in breast and colorectal tumor xenograft models in the current study. Additionally, capecitabine yielded good activity against A431 tumors. The combination of erlotinib and capecitabine, afforded at least additive activity against tumor growth in colon, breast and epidermal models.

The antitumor effects of chemotherapy are mediated through reversible GI cell cycle arrest, and a rapid apoptotic response (Kastan, M. B. (1997) Am. Soc. Clin. Oncol. Educational Book. W. B. Saunders, p15). It has been postulated that inhibition of HER1/EGFR-mediated proliferation and survival signals may lead to increased apoptosis in response to DNA damage in these tumors (Moyer J. D. et al. (1997) Cancer Res. 57:4838). In this way, the synchronous administration of erlotinib may potentiate the cytotoxic effects of capecitabine through increased apoptosis in the LoVo, KPL4 and A431 epithelial-derived human tumor models, and may therefore achieve supra-additive tumor inhibition compared with the constitutive single agents.

Anticancer agents, such as gefitinib, docetaxel, paclitaxel, cyclophosphamide and radiotherapy, have been shown to augment the effects of capecitabine through upregulation of tumor TP concentrations [Sawada, N., et al. (1998) Clin. Cancer Res. 4:1013; Sawada, N., et al (1999) Clin. Cancer Res. 5:2948; Fujimoto-Ouchi, K., et al. (2001) Clin. Cancer Res. 7:1079; Ishitsuka, H. (2000) Invest. New Drugs 18:343; Magne, N., et al. (2003) Clin. Cancer Res. 9:4735). TP plays a key role in the conversion of capecitabine to 5-FU in tumors, and the TP:DPD ratio has been shown to predict for capecitabine antitumor activity in human tumor xenograft models (Ishikawa, T., et al. (1998) Cancer Res. 58: 685). Erlotinib treatment affected the TP up-regulation in four out of the five models, whereas DPD levels were only increased in one of the tumor models in this study. Therefore, erlotinib, similar to other anticancer treatments, is likely to enhance the effects of capecitabine through positive effects on TP upregulation and TP:DPD ratios.

For clarification of TP and DPD up-regulation, immunohistochemical analysis was explored. The level of TP staining in tissue samples from erlotinib-treated mice was clearly stronger than in samples from vehicle-control animals, whereas the staining of DPD was similar between groups. Immunohistochemical methods are able to more clearly identify TP up-regulation compared with an ELISA. The reason for this distinction is that TP expression in tumor tissues is heterogenous and, therefore, is more adequately studied using immumohistochemistry, as ELISA methods assess enzyme levels in whole tissue.

In the A431 model the TP:DPD ratio increased 1.7 fold, suggesting that higher levels of 5-FU could be generated in the tumor, which may lead to an even greater increase in tumor-growth inhibition with no potentiation of bodyweight loss.

While most of the clinical trial data for erlotinib relate to its use in NSCLC, preliminary results from phase I/II studies have demonstrated promising activity for erlotinib and capecitabine/erlotinib combination therapy in patients with wide range of human solid tumor types, including CRC (Oza, M., et al. (2003) Proc. Am. Soc. Clin. Oncol. 22:196a, abstract 785) and MBC (Jones, R. J., et al. (2003) Proc. Am. Soc. Clin. Oncol. 22:45a, abstract 180). The current preclinical data confirm the significant antitumor activity of erlotinib against breast and colorectal tumor cells and demonstrate that the addition of capecitabine yields at least additive activity compared with the constitutive single-agent activities.

TABLE 1
Erlotinib/capecitabine combination: treatment administration
in animals bearing LoVo, A-431 and KPL-4 tumors

| Tumour | Erlotinib | Capecitabine | Erlotinib/capecitabine |
|---|---|---|---|
| model | (mg/kg/day) | (mg/kg/day) | combination (mg/kg/day) |
| LoVo | 100 | 359 | 100/359 |
| KPL-4 | 100 | 90 | 100/90 |
| A-431 | 100 | 359 | 100/359 |

TABLE 2
Tumor inhibition rates (%) following treatment for
approximately 2 weeks with erlotinib, capecitabine
or a combination of erlotinib and capecitabine

| | | | Erlotinib/capecitabine |
|---|---|---|---|
| Tumor model | Erlotinib | Capecitabine | combination |
| LoVo | 74* | 43* | 95 |
| KPL-4 | 71** | 36 | 88 |
| A-431 | 85** | 76** | 112 |

*p < 0.05 and
**p < 0.01 vs combination

TABLE 3
Erlotinib with capecitabine: toxicities in the LoVo
human colon cancer model (mean values ± SD)

| | | WBC count |
|---|---|---|
| Group | n | (×10⁶ cells/ml) |
| Control | 5 | 4.5 ± 0.6 |
| Erlotinib | 5 | 5.0 ± 0.5 |
| Capecitabine | 5 | 3.9 ± 0.5 |
| Combination | 5 | 3.9 ± 0.4 |

Occult blood tests and fecal observation were negative in all groups

TABLE 4
Effect of erlotinib (100 mg/kg/day) on tumor TP and DPD expression in
five human cancer xenograft models

| Tumor | | TP | | DPD | |
|---|---|---|---|---|---|
| model | n | Control | Erlotinib | Control | Erlotinib |
| A-431 | 5 | 408 ± 76 | 670 ± 126* | 71.8 ± 10.6 | 70.8 ± |
| | | | | | 12.8 |
| LoVo | 5 | 13.5 ± 2.5 | 24.0 ± 7.3* | 10.4 ± 3.3 | 24.0 ± |
| | | | | | 6.7* |
| HT-29 | 6 | 0.95 ± 0.11 | 1.16 ± 0.11* | 21.9 ± 3.6 | 25.0 ± |
| | | | | | 3.3 |
| KPL-4 | 5 | 45.8 ± 8.5 | 36.5 ± 10.8 | <2.0 | <2.0 |
| MAXF- | 5 | 11.0 ± 1.7 | 12.9 ± 1.1* | 3.36 ± 1.12 | 2.99 ± |
| 401 | | | | | 0.28 |

Values are means ± SD of enzyme specific activity (units/mg total protein). A unit of activity is defined as the generation of 1 μg 5-FU/hour for TP or the catabolism of 1 pmol 5-FU/minute for DPD.
*: p < 0.05 vs control group.

Incorporation by Reference

All patents, published patent applications and other references disclosed herein are hereby expressly incorporated herein by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A pharmaceutical composition comprising the EGFR kinase inhibitor erlotinib, or a salt thereof, and capecitabine, in a pharmaceutically acceptable carrier.

2. The pharmaceutical composition of claim 1, additionally comprising one or more other anti-cancer agents.

3. A composition in accordance with claim 3, wherein said other anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

4. A method for treating tumors or tumor metastases in a patient, comprising administering to said patient simultaneously or sequentially a therapeutically effective amount of a combination of the EGFR kinase inhibitor erlotinib and capecitabine.

5. The method of claim 4, wherein the patient is a human that is being treated for cancer.

6. The method of claim 4, wherein erlotinib and capecitabine are co-administered to the patient in the same formulation.

7. The method of claim 4, wherein erlotinib and capecitabine are co-administered to the patient in different formulations.

8. The method of claim 4, wherein erlotinib and capecitabine are co-administered to the patient by the same route.

9. The method of claim 4, wherein erlotinib and capecitabine are co-administered to the patient by different routes.

10. The method of claim 4, wherein erlotinib is administered to the patient by parenteral or oral administration.

11. The method of claim 4, wherein capecitabine is administered to the patient by parenteral or oral administration.

12. The method of claim 4, additionally comprising one or more other anti-cancer agents.

13. The method of claim 12, wherein the other anti-cancer agents are selected from an alkylating agent, cyclophosphamide, chlorambucil, cisplatin, carboplatin, busulfan, melphalan, carmustine, streptozotocin, triethylenemelamine, mitomycin C, an anti-metabolite, methotrexate, etoposide, 6-mercaptopurine, 6-thiocguanine, cytarabine, 5-fluorouracil, dacarbazine, an antibiotic, actinomycin D, doxorubicin, daunorubicin, bleomycin, mithramycin, an alkaloid, vinblastine, paclitaxel, a glucocorticoid, dexamethasone, a corticosteroid, prednisone, a nucleoside enzyme inhibitors, hydroxyurea, an amino acid depleting enzyme, asparaginase, leucovorin, and a folic acid derivative.

14. The method of claim 4, wherein the tumors or tumor metastases to be treated are selected from lung cancer, colorectal cancer, NSCLC, bronchioloalviolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, glioma, brain stem glioma, glioblastoma multiforme, astrocytoma, schwannoma, ependymoma, medulloblastoma, meningioma, squamous cell carcinoma and pituitary adenoma tumors or tumor metastases, including refractory versions thereof.

15. The method of claim 14, wherein the tumors or tumor metastases to be treated are colorectal cancer tumors or tumor metastases.

16. The method of claim 14, wherein the tumors or tumor metastases to be treated are breast cancer tumors or tumor metastases.

17. The method of claim 14, wherein the tumors or tumor metastases to be treated are epidermal cancer tumors or tumor metastases.

18. The method of claim 4, wherein the administering to the patient is simultaneous.

19. The method of claim 4, wherein the administering to the patient is sequential.

20. A method for the treatment of cancer, comprising administering to a subject in need of such treatment a first amount of the EGFR kinase inhibitor erlotinib, or a pharmaceutically acceptable salt thereof; and a second amount of capecitabine; wherein at least one of the amounts is administered as a sub-therapeutic amount.
